(19) United States
(12) Patent Application Publication
(10) Pub. No.: US 2005/0167999 A1
(43) Pub. Date: Aug. 4, 2005

(54) Safety device for holding a vial or ampule while extracting liquid contents with a syringe

(76) Inventor: Terry Jackman Beal, Copperas Cove, TX (US)

Correspondence Address: David O. Simmons, 7637 Parkview Circle, Austin, TX, 78731, US

(21) Appl. No.: 10/767,036

(22) Filed: Jan. 29, 2004

(51) Int. Cl. A61M 5/00

(52) U.S. Cl. 294/16

(57) ABSTRACT

A safety device comprising a pair of elongated arms and means connected to each one of the arms for enabling the arms to be moved between an open orientation and a closed orientation. Each one of the arms includes a first end, a second end and a first size container receiving recess positioned between the first and second ends. The means for enabling the arms to be moved between the open orientation and the closed orientation is connected to each one of the arms is adjacent its first end. The first size container receiving recess of each one of the arms jointly define a first size container receiving receptacle when the arms are in the closed orientation.

### FIELD OF THE DISCLOSURE

The disclosures made herein relate generally to devices for holding a vial or ampule while a syringe is used to extract liquid contents and, more particularly, to devices for holding a vile or ampule that allow a healthcare professional to be safe and sterile, and avoid accidental needle sticking when loading a syringe with liquid contents from a vile or ampule.

### BACKGROUND OF THE DISCLOSURE

Healthcare professional are often required to dispense medication (i.e., liquid contents extracted from a vial) via a syringe. Typically, this requires the professional to insert a needle of the syringe through a seal on the top of a vial, to load the syringe with medication, and then to inject the medication into the patient. Unfortunately, this procedure presents two well-known safety hazards.

First, loading a syringe presents the risk of accidental needle sticking—i.e., where a healthcare professional accidentally sticks himself or herself, or sticks another individual who is assisting with loading the syringe. This risk is particularly acute when one person holds the vile (or ampule) while another person inserts the needle into it or when the syringe is being loaded in a dim or dark environment such as an x-ray room. Significantly, it is estimated that between about 600,000 and about 800,000 healthcare professionals suffer needle stick accidents each year, (Minn. Med. 1995, 1765-8), and are put at risk for contracting diseases such as tuberculosis, streptococcal sepsis, Dengue fever, herpes, HDV, HGV, babesiosis, brucellosis, and Creutzfeldt-Jakob disease.

In recognition of this serious concern, Congress enacted the Needlestick Safety & Prevention Act of Nov. 11, 2000, which tasked OSHA with setting forth safety requirements to protect at-risk professionals and to encourage the use of needle safety devices. Consequently, the need exists for safety devices that help healthcare professionals avoid needle sticks when loading a syringe.

The second safety hazard occurs when a healthcare professional must load a syringe while in a sterile environment. In such a circumstance, loading a syringe presents the risk of contaminating either the healthcare professional or the environment. For example, when a doctor is performing surgery in an operating room, touching an object that has been sterilized—such as a vial containing medication—presents the risks of contamination through the operating field, and the spread of bacteria. Because it is important to avoid contact with unsterile objects under such circumstances, typically another individual must hold the vial while the doctor loads the syringe. However, the assistance of another person presents an added risk of contamination, especially when that person's hands come into close proximity to the syringe being loaded.

Accordingly, the need exists for a device that allows a doctor or other healthcare professional to load a syringe with medication in a manner that overcomes shortcomings associated with conventional approaches for loading a syringe with medication.

### SUMMARY OF THE DISCLOSURE

Embodiments of the inventive disclosures made herein are safety devices for holding a container (e.g., a via or ampule) while a syringe is used to extract liquid contents therefrom. Such safety devices aid healthcare professionals in avoiding accidental needle sticks and remaining sterile. In one embodiment, such a safety device comprises a pair of elongated arms and means connected to each one of the arms for enabling the arms to be moved between an open orientation and a closed orientation. Each one of the arms includes a first end, a second end and a first size container receiving recess positioned between the first and second ends. The means for enabling the arms to be moved between the open orientation and the closed orientation is connected to each one of the arms is adjacent its first end. The first size container receiving recess of each one of the arms jointly define a first size container receiving receptacle when the arms are in the closed orientation.

Accordingly, it is a principal object of the inventive disclosures made herein to provide a safety device that helps healthcare professionals avoid accidental needle sticks when loading a syringe.

It is another object of the inventive disclosures made herein to provide a safety device that helps healthcare professional avoid unsterilized objects when working in a sterile environment.

It is a further object of the inventive disclosures made herein to provide a safety device for holding a vial or ampule that can be easily sterilized for use in a sterile environment.

Still another object of the inventive disclosures made herein is to provide a safety device for holding a vial or ampule that is ergonomically configured for comfort and ease of use.

Yet another object of the inventive disclosures made herein is to provide a safety device for holding a vial or ampule that is inexpensive to manufacture, dependable and fully effective in accomplishing its intended purposes.

These and other objects of the inventive disclosures made herein will become readily apparent upon further review of the following specification and associated drawings.

### BRIEF DESCRIPTION OF THE DRAWINGS

FIGS. 1 and 2 depict a safety device in accordance with a first embodiment of the inventive disclosures made herein.

FIG. 3 depicts a safety device in accordance with a second embodiment of the inventive disclosures made herein, which includes compliant members at least partially defining container receiving receptacles.

FIG. 4 depicts a safety device in accordance with a third embodiment of the inventive disclosures made herein, which includes compliant members on surfaces that define container receiving receptacles.

### DETAILED DESCRIPTION OF THE DRAWINGS

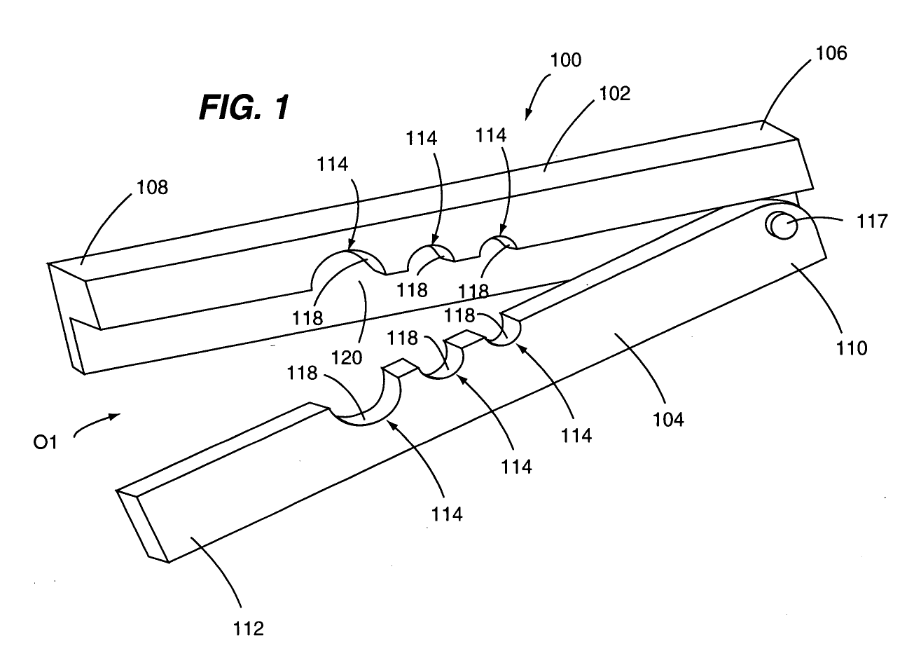

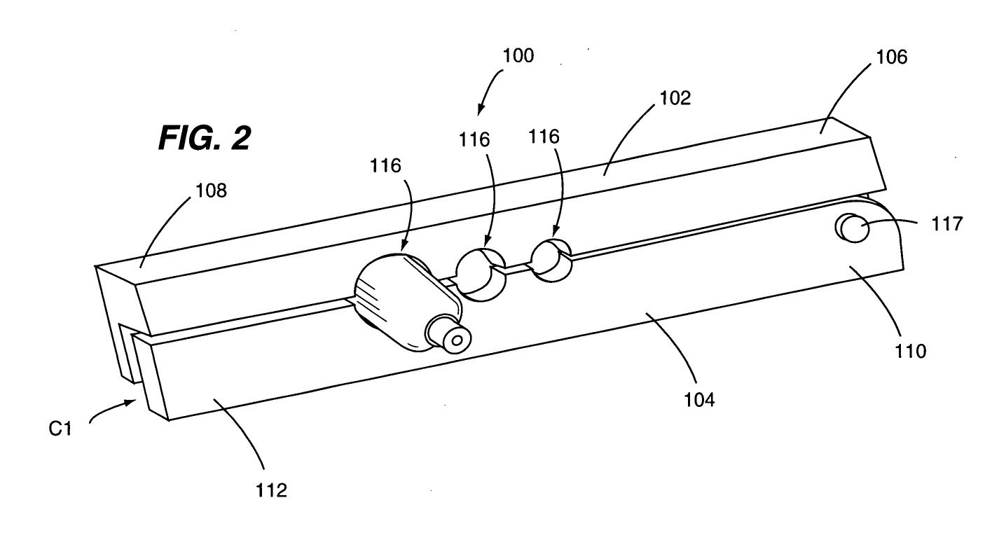

FIGS. 1 and 2 depict a safety device 100 in accordance with a first embodiment of the inventive disclosures made herein. The safety device 100 includes a first elongated arm 102 and a second elongated arm 104. It is contemplated herein that the arms (102, 104) may be formed from commercially available polymeric materials (e.g., polypropylene, nylon, etc) using known manufacturing approaches (e.g., injection molding, extrusion, etc.). The first elongated arm 102 is movably attached to the second elongated arm 104 for enabling the first and second arms (102, 104) to be moved between an open orientation O1 and a closed orientation C1. The first elongated arm 102 has a first end 106 and a second end 108. The second elongated arm 104 has a first end 110 and a second end 112.

Each one of the arms (102, 104) includes a plurality of container receiving recesses 114 (FIG. 1). As depicted, each one of the container receiving recesses 114 is a different size. Each one of the container receiving recesses 114 of the first elongated arm and the corresponding one of the container receiving recesses of the second elongated arm 104 represent mating portions of the arms (102, 104) that jointly define a plurality of container receiving receptacles 116 (FIG. 2) when the arms (102, 104) are in the closed orientation C1. The size of the container receiving receptacles 116 correspond to the size of the respective container receiving recesses 114. It is contemplated that each one of the arms (102, 104) may include a single container receiving recess (i.e., in an embodiment, not specifically shown).

As depicted in FIGS. 1 and 2, the first elongated arm 102 and the second elongated arm 104 are pivotally attached to each other at their respective first ends (106, 110) via a fastener 117 (e.g., a pin, rivet or the like). It is contemplated herein that a hinge (e.g., a discrete hinge or a living hinge) may be implemented for enabling the first elongated arm 102 and the second elongated arm 104 to be pivoted between the open orientation O1 and the closed orientation C1.

The container receiving recesses 114 each include semi-circular side wall 118 (i.e., of the corresponding container receiving recesses 114) and a rear wall 120. Other wall shaped such as a U-shaped wall is contemplated. As depicted, the rear wall 120 is comprised entirely by the first elongated arm 102. In another embodiment (not shown), the first elongated arm 102 and the second elongated arm 104 jointly comprise the rear wall 120. It is contemplated herein that the container receiving recesses 114 may be omitted from one of the arms (102, 104).

It should be understood that the ends of the elongated members (102, 104) are not discrete positions but rather regions that provide for hand gripping of the safety device 100. For example, the ends of the elongated members may be sized and/or shaped to permit hand gripping. It is contemplated herein that, in one specific embodiment, the ends of the elongated members (102, 104) include discrete gripping handles attached thereto.

It should be understood that the terms open orientation (e.g., open orientation O1) and closed orientation (e.g., O2) are relative orientations rather than specific positions. For example, an open orientation as referred to herein is an orientation that allows for a container to be positioned in one of the container receiving recesses 114 and a closed orientation as referred to herein is an orientation where the container is captured within a container receiving receptacle jointly defined between the first elongated arm 102 and the second elongated arm 104 (e.g., by corresponding container receiving recesses 114).

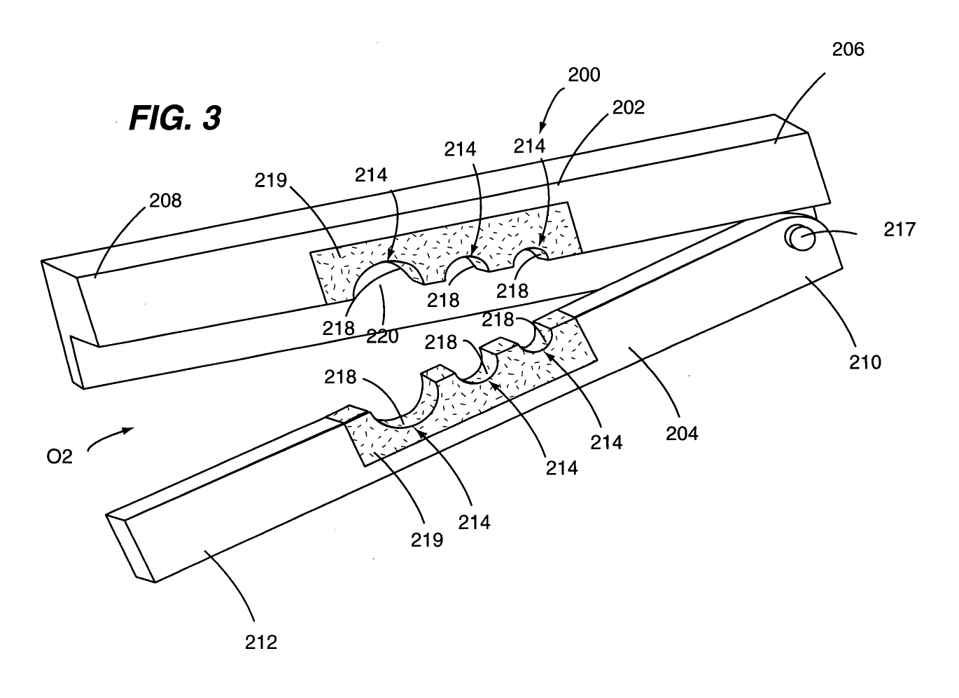

FIG. 3 depicts a safety device 200 in accordance with a second embodiment of the inventive disclosures made herein, which includes compliant members at least partially defining container receiving receptacles. The safety device 200 includes a first elongated arm 202 and a second elongated arm 204. The first elongated arm 202 is movably attached to the second elongated arm 204 for enabling the first and second arms (202, 204) to be moved between an open orientation O2 and a closed orientation (not specifically shown). The first elongated arm 202 has a first end 206 and a second end 208. The second elongated arm 204 has a first end 210 and a second end 212.

Each one of the arms (202, 204) includes a plurality of container receiving recesses 214. As depicted, each one of the container receiving recesses 214 is a different size. Each one of the container receiving recesses 214 of the first elongated arm and the corresponding one of the container receiving recesses of the second elongated arm 204 represent mating portions of the arms (202, 204) that jointly define a plurality of container receiving receptacles when the arms (202, 204) are in the closed orientation. The size of the container receiving receptacles corresponds to the size of the respective container receiving recesses 214.

The container receiving recesses 214 each include semi-circular side wall 218 (i.e., of the corresponding container receiving recesses 214) and a rear wall 220. A compliant member 219 at least partially defines the side walls 218 of the container receiving recesses 214 of each one of the arms (202, 204). Examples of a compliant member 219 include removable or integrally formed (e.g., co-molded) foam or elastomeric members. In addition to defining the container receiving recesses 214, the compliant member 219 serves to aid in gripping a container and in compensating for variations from a nominal container size (e.g., diameter).

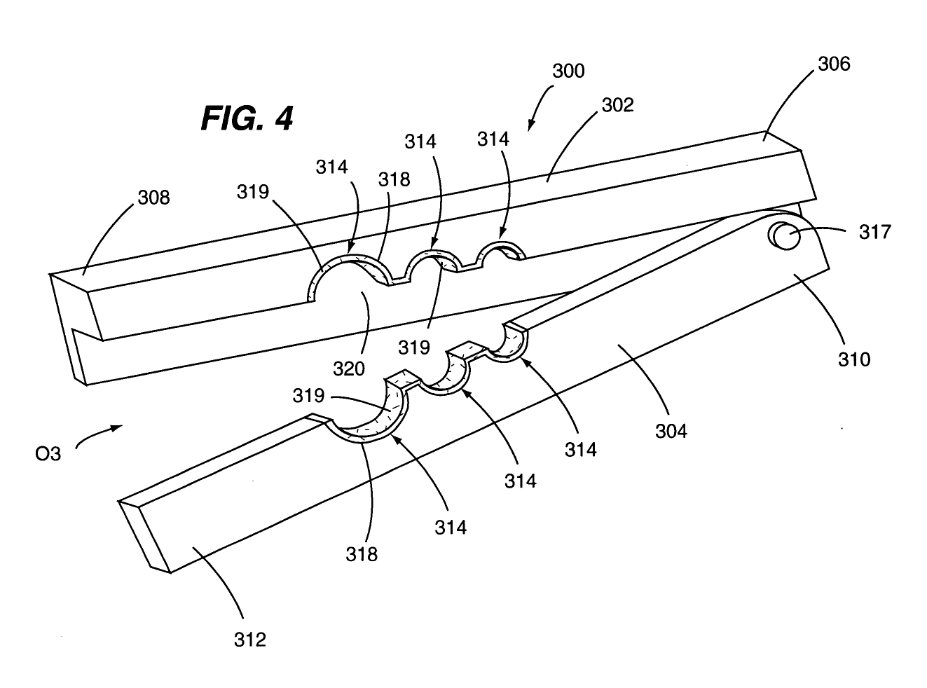

FIG. 4 depicts a safety device 300 in accordance with a third embodiment of the inventive disclosures made herein, which includes compliant members on surfaces that define container receiving receptacles. The safety device 300 includes a first elongated arm 302 and a second elongated arm 304. The first elongated arm 302 is movably attached to the second elongated arm 304 for enabling the first and second arms (302, 304) to be moved between an open orientation O3 and a closed orientation (not specifically shown). The first elongated arm 302 has a first end 306 and a second end 308. The second elongated arm 304 has a first end 310 and a second end 312.

Each one of the arms (302, 304) includes a plurality of container receiving recesses 314. As depicted, each one of the container receiving recesses 314 is a different size. Each one of the container receiving recesses 314 of the first elongated arm and the corresponding one of the container receiving recesses of the second elongated arm 304 represent mating portions of the arms (302, 304) that jointly define a plurality of container receiving receptacles when the arms (302, 304) are in the closed orientation. The size of the container receiving receptacles correspond to the size of the respective container receiving recesses 314.

The container receiving recesses 314 each include semi-circular side wall 318 (i.e., of the corresponding container receiving recesses 314) and a rear wall 320. The semicircular side walls 318 are formed in the arms (302, 304). A compliant member 319 (e.g., a foam or elastomeric member) is mounted on at least a portion of each semi-circular wall 318. The compliant member 319 serves to aid in gripping a container and in compensating for variations from a nominal container size (e.g., diameter).

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

1. A safety device for holding a container while extracting liquid contents therefrom with a syringe, comprising:
a first elongated arm having a first end and a second end; and
a second elongated arm having a first end and a second end; wherein:
the first elongated arm is movably attached to the second elongated arm for enabling said arms to be moved between an open orientation and a closed orientation;
mating portions of said arms jointly define at least one container receiving receptacle positioned between said ends when said arms are in the closed orientation; and
said at least one container receiving receptacle includes a surface configured for engaging side portions of a container disposed within said at least one container receiving receptacle and a surface configured for engaging a bottom portion of the container whereby the receptacle is configured for engaging the container at said bottom and side portions thereof.

2. The safety device of claim 1 wherein said arms are pivotally attached to each other at said first ends for enabling said arms to be pivoted between the open orientation and the closed orientation.

3. The safety device of claim 1 wherein said arms jointly define a plurality of different size container receiving receptacles positioned between said ends when said arms are in the closed orientation.

4. The safety device of claim 1 wherein:
said at least one container receptacle includes a side wall and a rear wall;
the surface configured for engaging side portions of the container is at least partially comprised by the side wall; and
the surface configured for engaging the bottom portion of the container is at least partially comprised by the rear wall.

5. The safety device of claim 4 wherein the rear wall is comprised entirely by one of said arms.

6. The safety device of claim 4 wherein the side wall is a semi-circular side wall.

7. The safety device of claim 1 wherein:
one of said mating portions include a compliant member that at least partially defines a side wall of said at least one container receiving receptacle; and
the surface configured for engaging side portions of the container is at least partially comprised by the side wall.

8. The safety device of claim 7 wherein the compliant member is removably mounted on one of said arms.

9. The safety device of claim 7 wherein the side wall is a semi-circular side wall.

10. A safety device for holding a container while extracting a liquid contents therefrom with a syringe, comprising:
a pair of elongated arms each including a first end, a second end and a first size container receiving recess positioned between the first and second ends; and
means connected to each one of said arms adjacent the first end for enabling said arms to be moved between an open orientation and a closed orientation;
wherein the first size container receiving recess of each one of said arms jointly define a first size container receiving receptacle when said arms are in the closed orientation; and
wherein the first size container receiving recess of at least one of said arms includes a surface configured for engaging side portions of a container disposed therein and a surface configured for engaging a bottom portion of the container whereby the first size container receiving recess of at least one of said arms is configured for engaging the container at said bottom and side portions thereof.

11. The safety device of claim 10 wherein said means is a hinge arrangement for enabling said arms to be moved between the open and closed orientations.

12. The safety device of claim 10 wherein:
each one of said arms further includes a second size container receiving recess positioned adjacent to the first size container receiving recess; and
the second size container receiving recess of each one of said arms jointly define a second size container receiving receptacle different than the first size container receiving receptacle when said arms are in the closed orientation.

13. The safety device of claim 10 wherein:
the first size container receiving recess of each one of said arms includes a side wall;
the first size container receiving recess of one of said arms includes a rear wall;
the surface configured for engaging side portions of the container is at least partially comprised by the side wall; and
the surface configured for engaging the bottom portion of the container is at least partially comprised by the rear wall.

14. The safety device of claim 10 wherein:
the first size container receiving recess of one of said arms includes a side wall comprised at least partially by a compliant member; and
the surface configured for engaging side portions of the container is at least partially comprised by the side wall.

15. The safety device of claim 14 wherein the compliant member is removably mounted on said one arm.

16. The safety device of claim 14 wherein the side wall is a semi-circular side wall.

17. A safety device for holding a container while extracting a liquid contents therefrom with a syringe, comprising:
a pair of elongated arms each including a first end, a second end and a plurality of different size container receiving recesses positioned between the first and second ends; and
a hinge arrangement disposed between said arms adjacent the first ends of said arms thereby enabling said arms to be moved between an open orientation and a closed orientation;
wherein said different size container receiving recesses of each one of said arms jointly define respective size container receiving receptacles when said arms are in the closed orientation; and
wherein said different size container receiving recesses of at least one of said arms each include a surface configured for engaging side portions of a respective container disposed therein and a surface configured for engaging a bottom portion of the respective container whereby said different size container receiving recesses of at least one of said arms are each configured for engaging the respective container at said bottom and side portions thereof.

18. The safety device of claim 17 wherein:
each one of said different size container receiving recesses includes a semi-circular side wall;
each one of said different size container receiving recesses of one of said arms includes a rear wall;
the surface configured for engaging side portions of the container is at least partially comprised by the semi-circular side wall; and
the surface configured for engaging the bottom portion of the container is at least partially comprised by the rear wall.

19. The safety device of claim 17 wherein:
each one of said different size container receiving recesses of one of said arms includes a semi-circular side wall comprised at least partially by a compliant member; and
the surface configured for engaging side portions of the container is at least partially comprised by the semi-circular side wall.

20. The safety device of claim 19 wherein the compliant member is removably mounted on said one arm.

21. The safety device of claim 20 wherein:
each one of said different size container receiving recesses of said one arm includes a rear wall; and
the surface configured for engaging the bottom portion of the container is at least partially comprised by the rear wall.
